(19) United States
(12) Patent Application Publication
(10) Pub. No.: US 2003/0109537 A1
(43) Pub. Date: Jun. 12, 2003

(54) Methods and materials for treating bone conditions

(76) Inventors: Russell T. Turner, Wabasha, MN (US); Sutada Lotinun, Rochester, MN (US); Peggy Backup, Wabasha, MN (US)

Correspondence Address: FISH & RICHARDSON P.C., 3300 DAIN RASCHER PLAZA, 60 SOUTH SIXTH STREET, MINNEAPOLIS, MN, 55402, US

(21) Appl. No.: 10/193,012

(22) Filed: Jul. 9, 2002

Related U.S. Application Data
(60) Provisional application No. 60/303,850, filed on Jul. 9, 2001.

(51) Int. Cl. A61K 31/519

(52) U.S. Cl. 514/259.31

(57) ABSTRACT

Methods and kits for treating bone conditions are described that use platelet-derived growth factor signaling antagonists.

### CROSS-REFERENCE TO RELATED APPLICATIONS

This application claims priority from U.S. Provisional Application Serial No. 60/303,850, filed Jul. 9, 2001.

### STATEMENT AS TO FEDERALLY SPONSORED RESEARCH

Funding for the work described herein was provided in part by the federal government, which may have certain rights in the invention.

### TECHNICAL FIELD

The invention relates to methods and materials for treating bone conditions. More specifically, the invention relates to using platelet-derived growth factor (PDGF) signaling antagonists to prevent or treat bone conditions.

### BACKGROUND

Parathyroid hormone (PTH) is a major physiological regulator of bone metabolism. Chronic elevation of PTH levels in humans, however, leads to a metabolic bone disease known as parathyroid bone disease. Osteitis fibrosa cystica, the most severe form of parathyroid bone disease, is rarely encountered in primary hyperparathyroidism, but frequently occurs in poorly managed renal osteodystrophy. Renal osteodystrophy occurs in patients with chronic renal failure and, in essence, is a disorder of bone remodeling. The impairment of the kidney to convert 25-hydroxyvitamin D₃ to 1α, 25-dihydroxyvitamin D₃ and to excrete phosphate results in hypocalcemia and phosphate retention, leading to a chronic increase in PTH secretion. The types of skeletal changes observed in chronic hyperparathyroidism depend on the severity and duration of the disease: (i) increased bone turnover, resulting in an increased risk for traumatic fractures, (ii) dissecting osteitis, tunneling trabeculae by osteoclasts with an excess of osteoid formation, (iii) osteitis fibrosa, bone resorption accompanied by fibrosis around the weakened trabeculae, and (iv) osteitis fibrosa cystica, replacement of marrow by fibrous tissue, microfractures and microhemorrhages with hemosiderin laden macrophages that often display multinucleated osteoclast-like giant cells resulting in a cystic brown tumor. The treatments currently used to manipulate this skeletal disease are vitamin D supplementation and partial parathyroidectomy, which relieves symptoms, but can lead to undesirable side effects, including adynamic bone disease.

### SUMMARY

The invention is based on the discovery that PDGF signaling antagonists can reduce the number of osteoclasts and reduce marrow fibrosis in an animal model of parathyroid bone disease that is induced by continuous PTH administration. As described herein, reducing the negative effects of PDGF on the processes of bone resorption and marrow fibrosis can aid in the treatment and prevention of bone conditions such as osteoporosis, hypercalcemia due to malignancy, renal osteodystrophy, and hyperparathyroidism.

In one aspect, the invention features a method for treating a bone condition in a mammal (e.g., a human or a rodent). The method includes administering to the mammal an amount of a PDGF signaling antagonist (e.g., receptor antagonist) effective to treat the bone condition and monitoring the bone condition in the mammal. The invention also features a method for preventing development of a bone condition in a mammal (e.g., a human or a rodent). The method includes administering to the mammal an amount of a PDGF signaling antagonist (e.g., receptor antagonist) effective to prevent the development of the bone condition and monitoring the mammal for development of the bone condition.

In either case, the bone condition can be a metabolic bone condition such as primary or secondary osteoporosis (e.g., postmenopausal osteoporosis, disuse osteoporosis, or senile osteoporosis), or parathyroid bone disease. A PDGF receptor antagonist can be triazolopyrimidine or a pharmaceutically acceptable salt thereof, (e.g., about 10 mg/kg/day to about 100 mg/kg/day of triazolopyrimidine or a pharmaceutically acceptable salt thereof). The monitoring step can include measuring calcium levels in a biological sample from the mammal, measuring levels of a marker of bone turnover in a biological sample from the mammal, or measuring bone mass and/or bone density in the mammal. The biological sample can be selected from the group consisting of blood, serum, plasma, bone, and urine. The marker of bone turnover can be selected from the group consisting of osteocalcin, bone specific alkaline phosphatase, type I C-terminal propeptide of type I collagen, deoxypyridinoline, and pyridinoline. Bone mass and bone density can be monitored using dual-energy absorptiometry or quantitative computed tomography.

In another aspect, the invention features a method of identifying a triazolopyrimidine derivative suitable for treating a bone condition. The method includes contacting a cell culture with the derivative in the presence of PDGF, and monitoring matrix protein production (e.g., osteocalcin production) or production and release of osteoclast stimulating cytokines (e.g., interleukin-6) in the cell culture. Stimulation of matrix protein production or inhibition of production and release of osteoclast stimulating cytokines in the cell culture indicates that the derivative is suitable for treating the bone condition. The cell culture can be a human bone cell culture such as a human fetal osteoblast line or a rodent bone cell culture such as a rat osteosarcoma line.

In yet another aspect, the invention features an article of manufacture that includes a PDGF signaling antagonist (e.g., triazolopyrimidine or a triazolopyrimidine derivative) or a pharmaceutically acceptable salt thereof and a package label or insert indicating that administration of the PDGF signaling antagonist is effective to treat a bone condition in a mammal.

In other embodiments, the invention features the use of a PDGF signaling antagonist such as triazolopyrimidine or a triazolopyrimidine derivative in the manufacture of a medicament for the treatment or prevention of a bone condition.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1A is a phosphorimage from an RNase protection assay depicting PDGF-A, L32 (a ribosomal protein), and GAPDH mRNA fragments from the tibial metaphysis of rats given vehicle alone, intermittent PTH, and continuous PTH. FIG. 1B is a bar graph depicting the quantitation of PDGF-A mRNA fragments normalized to L32 mRNA fragments. An “a” indicates a significance of p<0.05 compared with vehicle; “b” indicates a significance of p<0.05 compared with intermittent PTH. Data are presented as the mean±SEM.

FIG. 2A is a bar graph depicting the osteoblast surface to bone surface ratios (Ob.S/BS) in rats given vehicle, trapidil, continuous PTH, or continuous PTH plus trapidil. FIG. 2B is a bar graph depicting the osteoclast surface to bone surface ratios (Oc.S/BS) in rats given vehicle, trapidil, continuous PTH, or continuous PTH plus trapidil. An “a” indicates a significance of p<0.05 compared with vehicle; “b” indicates a significance of p<0.05 compared with trapidil; “c” indicates a significance of p<0.05 compared with PTH. Data are presented as the mean i SEM.

FIG. 3 is a bar graph depicting the fibrosis surrounding trabecular surface (% fibrotic perimeter) in rats given vehicle, trapidil, continuous PTH, or continuous PTH plus trapidil. An “a” indicates a significance of p<0.05 compared with vehicle; “b” indicates a significance of p<0.05 compared with trapidil; “c” indicates a significance of p<0.05 compared with PTH. Data are presented as the mean±SEM.

### DETAILED DESCRIPTION

In general, the invention provides methods for treating a bone condition, or preventing development of a bone condition, in a mammal that include administering a PDGF signaling antagonist (e.g., PDGF receptor antagonist) to the mammal. PDGF is a homo- or heterodimer of two polypeptide chains, PDGF-1 (PDGF-A) and PDGF-2 (PDGF-B), which show 56% homology and are linked by disulfide bonds. A gene on chromosome 7 (GenBank Accession No. X03795) encodes PDGF-A. PDGF-B is encoded by the c-sis protooncogene localized on chromosome 22 (GenBank Accession No. X02811). The PDGF-A homodimer binds only to its specific receptor (α), while the PDGF heterodimer and the PDGF-B homodimer bind to both the α and the β receptors. Without being bound by a particular mechanism, PDGF secreted within bone tissue in response to increased hormone levels such as PTH may induce growth and formation of fibroblasts and osteoclasts, the cells responsible for fibrosis and bone resorption, respectively. Administering PDGF signaling antagonists can prevent PDGF from interacting with PDGF receptors on fibroblasts and osteoblasts, which directly inhibits the growth of the former and which inhibits osteoclastic development and maturation indirectly. PDGF signaling antagonists such as triazolopyrimidine also can antagonize PDGF signaling by decreasing PDGF and PDGF receptor gene expression. As a result, marrow fibrosis and an increase in bone resorption are prevented.

PDGF Signaling Antagonists

Suitable PDGF antagonists interfere with the signaling activity of PDGF and can be a biological macromolecule such as an oligonucleotide or a polypeptide (e.g., an antibody), a chemical compound, a mixture of chemical compounds, or an extract isolated from bacterial, plant, fungal, or animal matter. Antagonists can interfere with the signaling activity of PDGF by preventing or reducing expression of PDGF, preventing or reducing expression of PDGF receptor, or by preventing or reducing the ability of PDGF to bind to its receptor. Non-limiting examples of PDGF signaling antagonists that can be used include triazolopyrimidine (also known as Trapidil or 5-methyl-7-diethylamino-S-triazol-(1,5α) pyrimidine), triazolopyrimidine derivatives such as 5,7 disubstituted 5-triazol-(1,5α) pyrimidines (e.g., AR12456, AR12463, AR12464, and AR12465), and pharmaceutically acceptable salts thereof. See, for example, Corsini et al., Pharmacol. Res., 21(5):521-531 (1989). Trapidil is thought to inhibit PDGF signaling via competitive binding to the PDGF receptor, and via reducing the expression of both PDGF and PDGF receptor. Trapidil is available commercially (e.g., from Rodleben Pharma GmbH, Rodleben, Germany).

Suitable oligonucleotides can be RNA or DNA based nucleic acids including chimeric mixtures, derivatives, and modified versions thereof. The oligonucleotide can be modified at the base moiety, sugar moiety, or phosphate backbone, for example, to improve stability of the molecule or to allow hybridization. A modified phosphate backbone can include, for example, phosphorothioate, phosphorodithioate, phosphoramidothioate, phosphoramidate, phosphordiamidate, methylphosphonate, alkyl phosphotriester, formacetel linkages, or analogs thereof. An oligonucleotide also can be a peptide nucleic acid, an uncharged nucleic acid derivative that contains a pseudopeptide backbone. Peptide nucleic acids can be produced using standard techniques. See, for example, U.S. Pat. No. 5,539,082.

An oligonucleotide can be an antisense oligonucleotide, e.g., complementary to at least a portion of the coding sequence or transcribed untranslated region of PDGF-A or the PDGF-α receptor. Antisense oligonucleotides can be full-length or less than full-length. Antisense oligonucleotides that are less than full-length are typically at least 6 nucleotides in length, e.g., from 6 to about 200 nucleotides in length. The term “complementary” refers to a sequence that is able to hybridize with the RNA, forming a stable duplex under normal in vivo conditions. The ability to hybridize depends on both the degree of complementarily and the length of the antisense nucleic acid. Generally, the longer the hybridizing nucleic acid, the more base mismatches with an RNA it may contain and still form a stable duplex. One skilled in the art can ascertain a tolerable degree of mismatch by use of standard procedures to determine the melting point of the hybridized complex. Administration of an effective amount of such antisense oligonucleotides would prevent expression of PDGF or its receptor, and inhibit PDGF signaling activity.

Oligonucleotides can be synthesized by standard methods known in the art, e.g., by use of an automated nucleic acid synthesizer (such as those commercially available from Biosearch, Applied Biosystems). Phosphorothioate oligonucleotides can be synthesized by the method of Stein et al., Nucl. Acids Res., 1988, 16:3209-3221. Methylphosphonate oligonucleotides can be prepared by use of controlled pore glass polymer supports, as described by Sarin et al., Proc. Natl. Acad. Sci. USA, 1988, 85(20):7448-7451.

In addition, ribozyme molecules can be designed to catalytically cleave PDGF (e.g., PDGF-A) or PDGF receptor transcripts, preventing expression of PDGF or PDGF receptor. Various ribozymes that cleave RNA can be used. For example, hammerhead ribozymes cleave RNAs at locations dictated by flanking regions that form complementary base pairs with the target RNA. The sole requirement is that the target RNA have the following sequence of two bases: 5′-UG-3′. The construction and production of hammerhead ribozymes is known in the art. See, for example, U.S. Pat. No. 5,254,678. Alternatively, RNA endoribonucleases such as the one that occurs naturally in Tetrahymena thermophila can be used. See, for example, U.S. Pat. No. 4,987,071.

Methods of Treating or Preventing Bone Conditions

Typically, a PDGF signaling antagonist is administered to a mammal such as a human patient that has been diagnosed with a bone condition. The term “bone condition” as used herein refers to any condition that increases osteoclast number, increases osteoclast activity, increases bone resorption, increases marrow fibrosis, or alters the calcium content of bone. Non-limiting examples of bone conditions include metabolic bone conditions such as renal osteodystrophy, primary forms of osteoporosis (e.g., postmenopausal and senile osteoporosis), and secondary forms of osteoporosis that develop as a result of an underlying disease state. For example, osteoporosis can develop in patients that have endocrine disorders such as hyperparathyroidism, hypo- and hyperthyroidism, hypogonadism, hypercalcaemia due to malignancy, pituitary tumors, type I diabetes, or Addison's disease. Neoplasias such as multiple myeloma and carcinomatosis also can lead to development of osteoporosis. In addition, gastrointestinal problems such as malnutrition, malabsorption, hepatic insufficiency, and vitamin C or D deficiencies, and chronic administration of drugs such as anticoagulants, chemotherapeutics, corticosteroids, anticonvulsants, and alcohol can lead to development of osteoporosis. Endocrine disorders, vitamin deficiencies, viral infections, and neoplasias also can lead to development of other bone conditions that can be treated with methods of the invention. For example, primary hyperparathyroidism or poorly managed renal osteodystrophy can lead to parathyroid bone disease.

PDGF signaling antagonists also can be administered prophylactically in patients at risk for developing a bone condition. For example, a PDGF signaling antagonist can be administered to patients undergoing glucocorticoid therapy to prevent steroid-induced osteoporosis from developing. A PDGF signaling antagonist also can be administered to post-menopausal women to prevent the development of osteoporosis.

In either case, an amount of PDGF signaling antagonist effective to treat or prevent the bone condition is administered to the patient. As used herein, the term “effective amount” refers to an amount of a PDGF signaling antagonist that reduces the deleterious effects of a bone condition, or prevents the development of deleterious effects of a bone condition, without inducing significant toxicity to the host. An effective amount of triazolopyrimidine can be at least about 10 mg/kg/day (e.g., 10-100, 10-20, 20-30, 30-40, 20-50, or 50-100 mg/kg/day) for a human patient. Effective amounts of other PDGF signaling antagonists can be determined by a physician, taking into account various factors that can modify the action of drugs such as overall health status, body weight, sex, diet, time and route of administration, other medications, and any other relevant clinical factors.

A PDGF signaling antagonist can be administered by any route, including, without limitation, oral or parenteral routes of administration such as intravenous, intramuscular, intraperitoneal, subcutaneous, intrathecal, intraarterial, nasal, or pulmonary absorption. A PDGF signaling antagonist can be formulated as, for example, a solution, suspension, or emulsion with pharmaceutically acceptable carriers or excipients suitable for the particular route of administration, including sterile aqueous or non-aqueous carriers. Aqueous carriers include, without limitation, water, alcohol, saline, and buffered solutions. Examples of non-aqueous carriers include, without limitation, propylene glycol, polyethylene glycol, vegetable oils, and injectable organic esters. Preservatives, flavorings, sugars, and other additives such as antimicrobials, antioxidants, chelating agents, inert gases, and the like also may be present.

For oral administration, tablets or capsules can be prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). Tablets can be coated by methods known in the art. Preparations for oral administration can also be formulated to give controlled release of the compound.

Nasal preparations can be presented in a liquid form or as a dry product. Nebulised aqueous suspensions or solutions can include carriers or excipients to adjust pH and/or tonicity.

Oligonucleotides and ribozymes can be delivered to a cell in vivo by a number of methods. For example, oligonucleotides can be injected directly into the tissue site, e.g., a tumor, or can be administered systemically. Alternatively, recombinant DNA constructs can be used to express oligonucleotides and ribozymes of the invention. For example, a vector can be introduced to a cell in vivo in a manner that allows the vector to be taken up by the cell, which can direct the transcription of the oligonucleotide or ribozyme. Vectors can remain episomal or can integrate into a chromosome, and are produced by standard recombinant DNA technology.

Monitoring Bone Conditions

Methods of the invention can include monitoring the mammal to, for example, determine if the bone condition is improving with treatment. Any method can be used to monitor a bone condition, including, without limitation, monitoring calcium levels, monitoring bone mass or bone density, monitoring bone turnover, monitoring changes in bone resorption, or monitoring changes in bone characteristics in a biological sample (e.g., blood, plasma, serum, urine, or bone) from the patient. Serum calcium levels can be determined by, for example, atomic absorption spectrophotometry (Cali et al., Clin. Chem., 19:1208-1213 (1973)), chelation with o-cresolphthalein complexone (Harold et al., Am. J. Clin. Pathol., 45:290-296 (1966)), or enzymatically with porcine pancreatic α-amylase orphospholipase D (Kimura et al., Clin. Chem., 42:1202-1205 (1996). Monitoring serum calcium levels is particularly useful in patients with bone conditions related to hyperparathyroidism, renal failure, or hypercalcemia due to malignancy. In such patients, a decrease in calcium levels over the course of treatment indicates that the bone condition is improving.

Bone turnover can be monitored by detecting the level of one or more biochemical markers of bone turnover, including osteocalcin, bone specific alkaline phosphatase, and type I C-terminal propeptide (CICP) of type I collagen. For example, the levels of osteocalcin can be detected in serum samples using commercially available immunoassays such as an enzyme-linked immunosorbent assay (ELISA) kit from Immuno Biological Laboratories (Hamburg, Germany) or Diagnostic Systems Laboratories, Inc. (Webster, Tex.) or a radioimmunoassay kit from Phoenix Pharmaceuticals, Inc. (Belmont, Calif.) or Biomedical Technologies Inc. (Stroughton, Mass.). Alternatively, Western blotting can be used. Monitoring osteocalcin levels is particularly useful for patients with a bone condition such as osteoporosis, including osteoporosis resulting from type I diabetes. In osteoporosis patients with high bone turnover, for example, caused by PTH excess, gonadal hormone deficiency, malignancy, or disuse, a decrease in osteocalcin levels over the course of the treatment indicates that the bone condition is improving. Bone specific alkaline phosphatase activity can be monitored in serum samples using commercially available immunoassay kits such as the ALKPHASE-B™ immunoassay kit (Quindel Corp., San Diego, Calif.). CICP, a biochemical indicator of collagen production, can be monitored in serum using an ELISA kit from Quindel Corp. (San Diego, Calif.).

Changes in bone resorption can be monitored by measuring levels of crosslinked collagen such as free deoxypyridinoline and free pyridinoline collagen crosslinks. Free deoxypyridinoline or free pyridinoline can be measured in urine samples using commercially available kits, e.g., an ELISA from Immuno Biological Laboratories (Hamburg, Germany). A decrease in the amount of free deoxypyridinoline or free pyridinoline over the course of the treatment indicates the bone condition is improving.

Bone mass and density also can be monitored in patients treated according to the methods of the invention. Bone mass can be measured in a patient using radiographic imaging techniques such as dual-energy absorptiometry. Bone density can be measured by quantitative computed tomography. An increase in bone mass or density over the course of the treatment indicates that the bone condition is improving in the patient.

Identifying PDGF Signaling Antagonists

The invention provides methods for identifying PDGF signaling antagonists (e.g., receptor antagonists or inhibitors of PDGF or PDGF receptor gene expression) that are suitable for treating or preventing one or more bone conditions in mammals. In vitro or in vivo models of bone disease can be used to identify suitable PDGF signaling antagonists, such as triazolopyrimidine derivatives. In vitro cell lines, including bone cell cultures such as human fetal osteoblast cell lines (hFOB) or rat osteosarcoma (ROS) cell lines, fibroblasts (NIH3T3 cells), or cultured explants from an animal model, can be used to identify suitable PDGF signaling antagonists. Such cells can be treated with a test compound over a period of time (e.g., days, weeks, or longer) then samples (e.g., cells and cell medium) can be collected and assayed for cell number, matrix protein production (e.g., collagen and osteocalcin production), or production and release of osteoclast stimulating cytokines (e.g., interleukin-6). As a control, the effect of the test compound can be compared with cultures treated with triazolopyrimidine (positive control) and to untreated cultures (negative control). If the effect of a particular test compound is similar to that of triazolopyrimidine, then that particular test compound may be suitable for treating a bone condition. Once a test compound is determined to be effective in vitro, the test compound can be tested in vivo. For example, a test compound can be administered to the rat model for parathyroid bone disease provided herein. Samples (e.g., blood, serum, urine, or bone) can be collected over a period of time and assayed. for markers that reflect the degree of parathyroid bone disease (e.g., serum calcium, serum and urine biochemical markers of bone turnover, osteoclast number, or fibrotic perimeter). The effect of the tested derivative can be compared to rat models treated with triazolopyrimidine as a positive control. If the effect of a particular derivative is similar to that of triazolopyrimidine, then that particular derivative may be effective for treating a bone condition in a mammal.

Articles of Manufacture

PDGF signaling antagonists described herein can be combined with packaging material and sold as an article of manufacture (e.g., a kit). Components and methods for producing articles of manufacture are well known. The PDGF signaling antagonist can be formulated as described herein for a particular route of administration, and can be packaged as a single dose or in multiple doses. Instructions describing how the PDGF signaling antagonist can be used to treat bone conditions may be included in such kits as a package insert. The package insert also can include examples of bone conditions that can be treated as well as suggested routes of administration, formulations, dosages, and methods of monitoring particular bone conditions to evaluate treatment.

The invention will be further described in the following examples, which do not limit the scope of the invention described in the claims.

### EXAMPLES

### Example 1

### Materials and Methods:

Induction of parathyroid bone disease. Three month-old female Sprague-Dawley rats (Harlan Sprague-Dawley, Inc., Indianapolis, Ind.) were randomly divided into 3 groups, with 5 rats per group. In group 1 (pulsatile or intermittent PTH), each rat received 80 μg/kg/day human PTH (1-34) (hPTH) in vehicle (150 mM NaCl, 1 mM HCl and 2% heat-inactivated rat serum) by subcutaneous (s.c.) injection once daily for 7 days. In group 2 (vehicle), an osmotic pump (Alza Corp., Mountainview, Calif.) that delivered vehicle alone at a rate of 1 μl/hr for 7 days was implanted in each rat. In group 3 (continuous PTH), an osmotic pump that delivered 40 μg/kg/day hPTH at a rate of 1 μl/hr for 7 days was implanted in each rat. On day 8, all rats were anesthetized with ketamine (50 mg/kg): xylazine HCl (5 mg/kg) and sacrificed by decapitation. Both tibiae were removed from each rat. Right tibiae were fixed by immersion in 70% ethanol then processed for bone histology to verify the appearance of peritrabecular fibrosis. Left tibiae were frozen in liquid N₂ and stored at −80° C. until processed for RNA isolation.

Isolation of RNA. Frozen proximal tibial metaphyses were individually homogenized in guanidine isothiocyanate using a Spex freezer mill (Industries, Inc., Edison, N.J.). Total RNA was extracted from the homogenate using a modified organic solvent method. See Chomczynski et al., Anal. Biochem., 162:156-159 (1987). Isolated RNA yields were determined spectrophotometrically at 260 nm using standard methods.

cDNA microarray analysis. cDNA probes were generated by reverse transcription (Superscript II, Life Technologies, Rockville, Md.) using 1 μg total RNA isolated from the proximal tibial metaphysis of rats in groups 1, 2, and 3. First-strand cDNA probes were primed by the addition of oligo dT and subsequently labeled with [α-³³P]dCTP (ICN Radiochemicals Costa Mesa, Calif.). The labeled probes then were purified by passage through a Sephadex G-50 DNA Grade Column (Amersham Pharmacia Biotech AB, Uppsala, Sweden). The purified probes were hybridized to a rat genefilter microarray containing 5531 genes (GF 300; Research Genetics, Huntsville, Ala.). according to the manufacturer's recommended protocol. A micro-array system from Affymetrix, which contains 8500 genes, also was used. After hybridization, the array was washed and wrapped with plastic wrap before placing in a phosphor imaging cassette containing a Cyclone Storage Phosphor Screen (Packard, Downers Groves, Ill.). After 24 hours, the screen was imaged and the resulting images analyzed using Pathways 2.01 software to compare the signal intensities of spots.

RNase protection assay. Steady state mRNA levels for PDGF-A and other growth factors were determined using an RNase protection assay kit according to the manufacturer's protocol (Pharmingen, San Diego, Calif.). Quantitation of protected RNA fragments was performed by PhosphoImager analyses and normalized to glyceraldehyde-3-phosphate dehydrogenase (GAPDH) and the ribosomal structural protein L32.

Treatment protocol. Three month-old female Sprague-Dawley rats were divided into 4 groups. In group 1 (n=9), osmotic pumps that delivered vehicle alone at a rate of 1 μl/hr for 7 days were implanted in each rat. Rats in group 2 (n=10) received 40 mg/kg/day triazolopyrimidine (Trapidil; Rodleben Pharma GmbH, Rodleben, Germany) by s.c. injection once daily for 7 days. In groups 3 and 4 (n=8 and 10, respectively), osmotic pumps were implanted in each rat that delivered 40 μg/kg/day hPTH at a rate of 1 μl/hr for 7 days. Each rat in group 4 also received 40 mg/kg/day trapidil by s.c. injection once daily for 7 days. The trapidil dosage was estimated based on inhibition studies of trapidil on several types of cells in the rat. See, for example, Futamura et al., Nephron, 81:428-433 (1999), Tiell, Artery, 12:33-50 (1983), and Gocer et al., Neurol. Res., 20:365-373 (1998). Tetracycline (20 mg/kg, Sigma Chemical Co., St. Louis, Mo.) and calcein (20 mg/kg, Sigma) fluorochrome labels were injected at the base of the tail on day 0 (tetracycline) and day 6 (calcein). On day 8, all rats were anesthetized and blood was collected by cardiac puncture. The blood samples were used to determine serum chemistry and PTH levels. Following blood collection, the rats were sacrificed by cervical dislocation, and tibiae were surgically removed and fixed in 70% ethanol for bone histomorphometry.

Serum chemistry and PTH. Total serum calcium, phosphate, and magnesium levels were measured in rat blood samples by Central Clinical Laboratory Research at the Mayo Clinic using automated procedures. Serum PTH was measured using an immunoradiometric assay for rat PTH (Immunotopics International, LLC, San Clementa, Calif.) that has approximately 100% cross-reactivity to human PTH.

Bone histomorphometry. Proximal metaphyses were dehydrated in a graded series of ethanol, then infiltrated and embedded in methymethacrylate (Fisher Scientific, Fair Lawn, N.J.). After embedding, 5 μm sections were cut using a microtome (Reichert-Jung Model 2065, Heidelberg, Germany), and the sections were mounted unstained. Dynamic histomorphometric cancellous bone measurements were made in the unstained sections using fluorescent microscopy to detect the injected tetracycline and calcein markers. After obtaining the cancellous bone measurements, consecutive sections were stained with toluidine blue for bone cell and peritrabecular fibrosis measurements using light microscopy. A standard sampling site of 2.8 mm was located in the secondary spongiosa of the metaphysis at 1.5 mm distal to the growth plate.

All histomorphometric measurements were made with an Osteomeasure image analysis system (OsteoMetrics, Atlanta, Ga.) coupled to a photomicroscope and personal computer. All parameters were calculated according to standardized nomenclature. See, for example, Parfitt et al., J. Bone Miner. Res., 2:595-610 (1987). Bone volume was defined as the percentage of tissue volume consisting of cancellous bone. Tetracycline and calcein labels were determined as the percentage of bone perimeter labeled with fluorochrome. Mineral apposition rate (MAR) was defined as the average width between tetracycline and calcein label divided by interlabel time of 6 days. Bone formation rate (BFR) was defined as the product of MAR and the calcein label perimeter, and was expressed per bone surface (BFR/BS), bone volume (BFR/BV), or tissue volume (BFR/TV). Osteoblast surface was defined as a palisade of large basophilic cuboidal cells directly lying on top of the osteoid, and was expressed as a percent of bone perimeter. Osteoclast surface was defined as the bone perimeter lined by multinucleated cells regardless of the presence of erosion. Fibrotic perimeter was defined as the bone perimeter lined by multilayers of fibroblasts.

Statistical analysis. Multiple group comparisons were determined using one-way analysis of variance (one-way ANOVA) with statistical significance at P<0.05. Differences between pairs of groups were compared by the Fisher's protected least significant difference post-hoc test. In therapeutic studies, two-way analysis of variance (two-way ANOVA) was performed to determine significant effects of PTH and trapidil, or interactions between PTH and trapidil.

### Example 2

### A Rat Model for Parathyroid Bone Disease

A rat model of hyperparathyroidism (HPT) was developed as described in Example 1, and compared to intermittent PTH treatment. PTH results in major changes in bone metabolism in less than 1 week and these short-term changes accurately predict the long-terms effects of the hormone. To induce an anabolic course of PTH action, human recombinant PTH was administered s.c. (80 μg/kg/d). PTH resulted in an upregulation of mRNA levels for bone matrix proteins (e.g., type I collagen, osteonectin, and osteocalcin) within 16 hours, an increase in ³H-proline incorporation into bone matrix proteins within 24 hours, and an increase in the number of fully mature osteoblasts within 3 days. In contrast, no increase in osteoclast number was noted.

To mimic HPT, hPTH was infused continuously at the same dose rate (80 μg/kg/d) using a s.c. implanted osmotic pump. Subcutaneous PTH had no effect on serum calcium levels but continuous release caused severe hypercalcemia and weight loss, which was deemed unacceptable. Reducing the dose rate of continuous PTH infusion to 40 μg/kg/d greatly reduced systemic side effects without preventing the detrimental skeletal effects of continuous PTH. These changes, which were similar to that of HPT patients, included extensive peritrabecular fibrosis, osteomalacia, increased bone formation, and focal bone resorption. In addition, bone formation in rats treated continuously or intermittently with PTH had similar increases in bone formation after 1 week. Cancellous osteopenia was not observed. Table 1 summarizes the effects of pulsatile and continuous PTH on bone histomorphometry in rats. Based on histological examination of osteitis fibrosa in HPT patients, it appears that the close association of the fibroblasts with bone surfaces indicates that continuous PTH results in the local release of paracrine factors that are chemotactic to fibroblasts and that stimulate their proliferation. The time course of PTH action in rats indicates that extensive marrow fibrosis precedes increased bone resorption.

TABLE 1
Comparison of pulsatile and continuous PTH
on bone histomorphometry in rats

| | Pulsatile | |
|---|---|---|
| Measurement | PTH | Continuous PTH |
| Osteoblast perimeter | ↑^{a} | ↑^{a} |
| Bone formation rate | ↑^{a} | ↑^{a} |
| mRNA levels for one matrix | ↑^{a} | ↑^{a} |
| proteins | | |
| Osteoclast perimeter | [symbol] ^{a} | ↑^{b} |
| Marrow fibrosis | Not observed^{a} | Extensive^{b} |
| Osteoid surface | Minimal^{a} | Extensive^{b} |

Measurements were performed at the proximal tibial diaphysis.
Increased compared to untreated control, ↑;
no change, [symbol] ;
same superscript means no differences between pulsatile and continuous PTH;
different superscript means pulsatile and continuous PTH differ (p < .05).

The relationship between skeletal abnormalities and the duration of the PTH pulse was defined by programming the implantable osmotic pumps to deliver the same quantity of PTH over different intervals. A 1 hour pulse induced a skeletal response similar to daily (intermittent) s.c. administration. By contrast, detrimental side effects were observed following administration of PTH using daily pulses as short as 2 hours. These detrimental side effects increased with pulse duration to have the same effect as continuous PTH with pulses lasting 6 hours. It appears that the duration of the PTH pulse required to increase bone formation without having detrimental side effects is very brief.

³H-thymidine autoradiography was performed to determine the role of cell proliferation in contributing to the increases in osteoblasts and fibroblasts following continuous administration of PTH. ³H-thymidine was infused continuously for the entire 1-week duration of PTH treatment in order to label all proliferating cells. Osteoblasts induced by continuous administration of PTH were unlabeled, indicating that they were derived by modulation rather than proliferation. In contrast to osteoblasts, most of the peritrabecular fibroblasts induced by continuous infusion with PTH were labeled with ³H-thymidine, indicating that these cells had progressed through the cell cycle. Thus, it seems that PTH-induced osteoblasts and fibroblasts originate by different cellular pathways.

The charts of 605 patients diagnosed with hyperparathyroidism (HPT) who had iliac crest bone biopsies were reviewed to examine the type and frequency of histomorphometric abnormalities. The results of this review are presented in Table 2. Bone formation was not measured in almost half of the patients because the fluorochrome labels were too diffuse to distinguish double labels. A similar phenomenon was observed in some histologic sections from the rats continuously infused with PTH. Rapid bone matrix deposition with delayed mineralization produces diffuse fluorochrome labeling. Thus, bone matrix synthesis in patients with HPT and rats treated with continuous PTH may be underestimated.

In spite of patient heterogeneity, 90% of HPT patients had marrow fibrosis (osteitis fibrosa). Other common abnormalities included excess osteoid and increased indices for bone resorption (eroded perimeter and osteoclast number). Increased bone formation was observed in about half of the patients and a small (11%) subgroup had decreased bone formation and a mineralizing defect (osteomalacia). Cancellous osteopenia was uncommon in these HPT patients indicating that the increase in bone resorption was focal, rather than generalized, or alternatively, that it is generally compensated for by increased bone formation. Analysis of the biopsies confirmed the validity of the rat model for HPT.

TABLE 2
Frequency of skeletal abnormalities identified in iliac crest bone
biopsies from patients diagnosed with HPT

| Measurement | Increased | Unchanged | Decreased |
|---|---|---|---|
| Marrow fibrosis | 90% | 10% | *NA |
| Osteoid surface | 86% | 13% | 1% |
| Bone formation rate | 52% | 37% | 11% |
| Eroded perimeter | 75% | 23% | 2% |
| Osteoclast surface | 89% | 8% | 3% |
| Bone volume | 8% | 82% | 10% |

Changes are compared to a healthy group of volunteers.
Increase, Z score ≧ 2;
decreased Z score ≦ −2;
unchanged, Z score < 2 and > −2.
*NA, not applicable because marrow fibrosis not detected in health volunteers.

### Example 3

### Identifying PTH-Regulated Genes in a Rat Model for HPT

Candidate genes associated with peritrabecular fibrosis were identified with cDNA microarrays containing 5531 or 8500 genes as described in Example 1. Approximately 14% of the total genes measured were differentially expressed by at least 2.5-fold between pulsatile and continuous PTH-treated groups. More specifically, at a confidence level of p<0.05, gene expression analysis of the 8500 rat genes (Affymetrix system) demonstrated that 3.6% of the genes were regulated by intermittent administration of PTH and 10.4% by continuous administration. Of the regulated genes, 158 were unique to intermittent administration of PTH and 759 to continuous administration of PTH. An additional 158 genes were common to both treatments.

Classification of regulated genes by pathways and function identified similarities and differences. Protein cleavage and degradation was the pathway most represented in genes unique to continuous administration of PTH and included components of the ubiquitin-proteosome degradation complex, as well as metalloproteases and their inhibitors. Based on functional classification, genes uniquely regulated by continuous administration of PTH encoded many 7-transmembrane/G protein-coupled receptors, and integral membrane proteins. Genes common to both intermittent and continuous administration of PTH included many integral membrane proteins and extracellular matrix proteins.

The list of candidate genes was examined to determine if any of the genes encoded growth factors. More specifically, it was determined if the differentially expressed genes were growth factors produced on or near bone surfaces, were chemotactic to fibroblasts, were able to stimulate fibroblast proliferation, or could induce bone resorption. Candidate genes that met at least one of the criteria are listed in Table 3. One of the candidate genes, PDGF, a known mitogenic and chemotactic factor for fibroblasts, met all these criteria.

TABLE 3
Candidate genes for cytokines and their receptors that were
differentially expressed by continuous PTH by gene microarray

| Gene | Fold Change | Significance |
|---|---|---|
| PDGF-A | 85 | P < .02^{a} |
| BMP-1 | 1.3 | P < .001 |
| BMP-6 | 1.6 | P = .05 |
| BMP-3 | 19 | P = .04 |
| FGF | 5.1 | P = .002 |
| PTH receptor | 3.8 | P = .05 |
| FGF receptor-1 | 2.7 | P <= .0005 |
| TGF-β₃ | 1.9 | P = .01 |
| COX2 | 1.8 | P = .001 |
| PGE₂ receptor EP₃ | 1.7 | P = .03 |
| TGF-_{β1} | 1.5 | P = .02 |
| IL-12p40 | 1.3 | P = .02 |
| IL-6 receptor | −1.2 | P = .03 |

^{a}n = 5

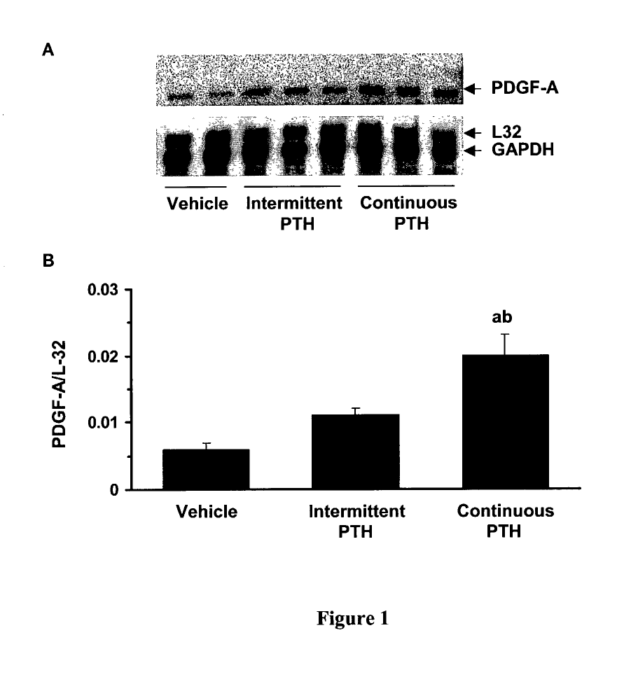

RNase protection assays were performed as described in Example 1, for PDGF-A, BMP-1, BMP-6, BMP-3, and TGF-β₁ to verify the microarray data. PTH-induced regulation of mRNA levels was confirmed for BMP-1, -3, and -6, as well as TGF-β. FIG. 1A depicts the RNase protection assay results for PDGF-A. Pulsatile PTH had no effect on steady state mRNA levels for PDGF-A, whereas continuous PTH resulted in a significant 3.3-fold increase in the mRNA levels for PDGF-A (FIG. 1B). These data demonstrated that PDGF-A mRNA is differentially regulated by continuous administration of PTH compared with pulsatile administration of PTH. Upon examining the time course of the mRNA expression, it was found that the increase in PDGF mRNA levels preceded the skeletal abnormalities induced by PTH.

### Example 4

### Administering a PDGF Receptor Antagonist Decreases PTH-Induced Marrow Fibrosis and Osteoclast Resorption

Based on the findings described in Example 3, agents that block PDGF-A binding to its cell-surface receptor (i.e., PDGF receptor antagonists) could be potential therapeutic agents for PTH-induced bone conditions. To test this hypothesis, groups of rats were given vehicle, continuous PTH, trapidil, or continuous PTH and trapidil as described in Example 1. Serum and bone samples were collected after 7 days and analyzed.

Serum chemistry analysis revealed that trapidil alone (Trapidil) had no effect on serum calcium, phosphorus, magnesium, or PTH levels compared to vehicle (Table 4). Continuous PTH (PTH) induced hypercalcemia and hyperparathyroidism. Rats that received trapidil in addition to continuous PTH (PTH+Trapidil) exhibited a significant reduction in hypercalcemia (51%, p<0.05) compared to continuous PTH (Table 4).

TABLE 4
Serum chemistry data

| | Vehicle | Trapidil | PTH | PTH + Trapidil | Two-way ANOVA | | |
|---|---|---|---|---|---|---|---|
| Parameters | (n = 9) | (n = 10) | (n = 8) | (n = 10) | PTH | Trapidil | Interaction |
| Calcium | 10.26 ± 0.07 | 10.10 ± 0.05 | 11.65 ± 0.28^{ab} | 10.94 ± 0.32^{abc} | P < 0.001 | P < 0.05 | NS |
| (mg/dl) | | | | | | | |
| Phosphorus | 7.89 ± 0.27 | 7.33 ± 0.29 | 7.95 ± 0.48 | 7.46 ± 0.29 | NS | NS | NS |
| (mg/dl) | | | | | | | |
| Magnesium | 2.34 ± 0.05 | 2.40 ± 0.05 | 2.49 ± 0.04 | 2.46 ± 0.07 | NS | NS | NS |
| (mg/dl) | | | | | | | |
| PTH (pg/ml) | 43.29 ± 7.58 | 51.98 ± 8.21 | 98.27 ± 11.12^{ab} | 118.49 ± 34.16^{ab} | P < 0.05 | NS | NS |

Data represent mean ± SEM.
^{a}, P < 0.05 compared with vehicle;
^{b}, P < 0.05 compared with trapidil;
^{c}, P < 0.05 compared with PTH.

Fluorochrome-based histomorphometric analyses of tibial metaphyseal sections revealed that neither trapidil nor PTH treatment had any effect on cortical bone histomorphometry. Continuous PTH stimulated cancellous bone formation, whether expressed per bone surface, bone volume, or tissue volume. This was due to an increase in osteoblast number as deduced from the increased calcein labeled surface (Table 5). The changes in cancellous bone are relevant to metabolic bone diseases including, for example, postmenopausal osteoporosis, disuse osteoporosis, hypercalcemia due to malignancy, hyperparathyroidism, and renal osteodystrophy. The fluorochrome-based analyses were confirmed by measurement of PTH-increased osteoblast surface. Continuous PTH slightly increased the rate of mineral apposition, an index of osteoblast activity, and was found to be significant upon two-way ANOVA. Trapidil had no significant effect on measurements related to the PTH-induced increase in bone formation.

TABLE 5
Histology data

| | Vehicle | Trapidil | PTH | PTH + Trapidil | Two-way ANOVA | | |
|---|---|---|---|---|---|---|---|
| Parameters | (n = 9) | (n = 10) | (n = 8) | (n = 10) | PTH | Trapidil | Interaction |
| BV/TV (%) | 24.89 ± 0.94 | 24.83 ± 0.87 | 27.25 ± 1.14 | 23.79 ± 0.86 | NS | NS | NS |
| Tetracycline | 7.86 ± 0.94 | 9.65 ± 1.76 | 5.65 ± 2.48 | 6.91 ± 1.71 | NS | NS | NS |
| label (%) | | | | | | | |
| Calcein label | 18.63 ± 2.22 | 18.32 ± 1.39 | 48.08 ± 5.35^{ab} | 43.24 ± 4.42^{ab} | P < 0.001 | NS | NS |
| (%) | | | | | | | |
| MAR | 1.09 ± 0.05 | 1.06 ± 0.04 | 1.29 ± 0.08 | 1.18 ± 0.07 | P < 0.05 | NS | NS |
| (μm/day) | | | | | | | |
| BFR/BS | 0.29 ± 0.03 | 0.30 ± 0.03 | 0.71 ± 0.10^{ab} | 0.59 ± 0.06^{ab} | P < 0.001 | NS | NS |
| (μm³/μm²/day) | | | | | | | |
| BFR/BV (%) | 1.04 ± 0.11 | 0.99 ± 0.08 | 2.30 ± 0.31^{ab} | 2.06 ± 0.18^{ab} | P < 0.001 | NS | NS |
| BFR/TV (%) | 0.26 ± 0.03 | 0.25 ± 0.02 | 0.63 ± 0.09^{ab} | 0.49 ± 0.05^{ab} | P < 0.001 | NS | NS |

Data represent mean ± SEM.
^{a}, P < 0.05 compared with vehicle;
^{b}, P < 0.05 compared with trapidil;
^{c}, P < 0.05 compared with PTH.
Abbreviations:
bone volume per tissue volume (BV/TV),
mineral apposition rate (MAR),
bone formation rate (BFR) expressed per bone surface (BS),
bone volume (BV),
and tissue volume (TV).

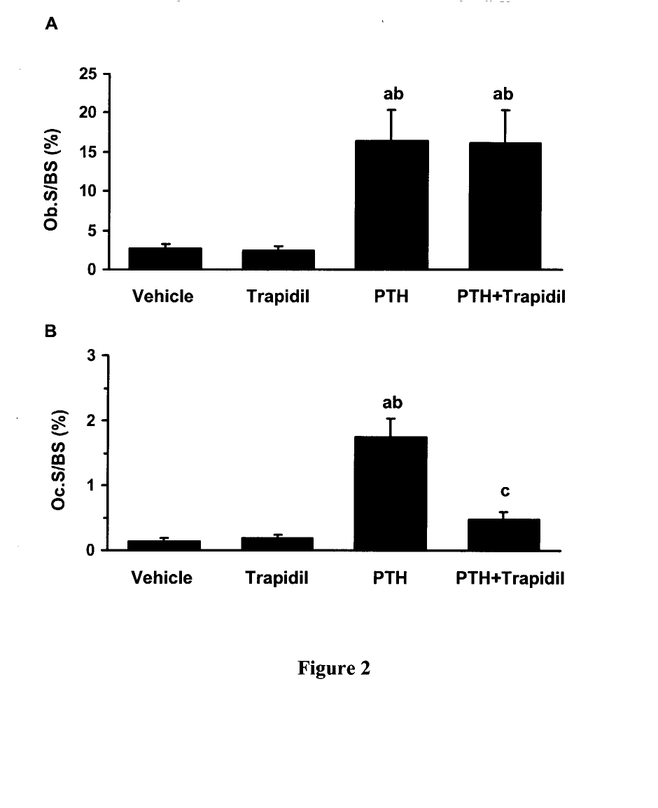

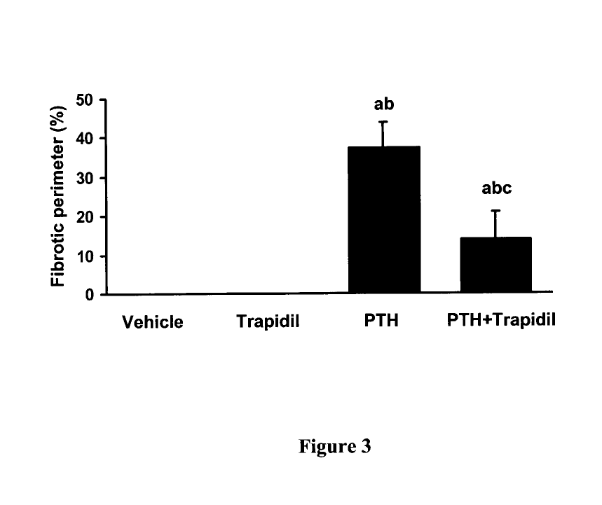

Continuous PTH increased osteoblast surface, and trapidil did not reverse this effect (FIG. 2A). Continuous PTH also increased osteoclast-lined surface, suggesting that bone resorption was increased (FIG. 2B) and induced extensive peritrabecular fibrosis (FIG. 3). Trapidil decreased PTH-induced osteoclast-lined surface (i.e., osteoclast perimeter) and peritrabecular fibrosis by 73 and 63%, respectively. Two-way ANOVA confirmed an interaction between PTH and trapidil on osteoclast-lined and peritrabecular fibrotic surface. The marrow area replaced by fibrotic tissue was reduced to an even greater extent by trapidil. These data demonstrate that trapidil reduces skeletal pathologies induced by continuous administration of PTH. The effect of trapidil depended upon PTH being present, i.e., trapidil blocked the effects of PTH, but had no effect on its own.

Immunohistochemistry for PDGF-A was performed on bone samples from PTH-treated, PTH+trapidil treated, and vehicle control animals. PTH increased the number of PDGF-A positive mast cells, whereas trapidil prevented the increase. Mast cells stained intensely for PDGF-A peptide in PTH-treated rats, but not in untreated animals.

The effects of trapidil on gene expression were evaluated using micro-arrays and RNase protection assays (Table 6). Candidate genes that may mediate the effects of PDGF were examined. RNA from proximal tibia metaphysis of rats treated with PTH was compared to RNA from animals treated with PTH and trapidil. Trapidil prevented the expected increases in BMP-2, -3, and -6, PDGF-A, and PDGF receptor induced by continuous PTH. Trapidil has no effect on other PTH-induced changes including the increases in BMP-4, TGF-β₁, IFN-γ, and TNF-α. Trapidil did not alter the expression of a panel of growth factor genes that were not regulated by PTH. These data support the hypothesis that PDGF signaling is essential for selected actions of PTH.

TABLE 6
Effects of PTH and Trapidil on steady-state mRNA levels
for selected cytokines and growth factors

| Gene | PTH^{a} | PTH + Trapidil^{b} |
|---|---|---|
| BMP-2 | ↑ | ↓ |
| BMP-3 | ↑ | ↓ |
| BMP-4 | [symbol] | [symbol] |
| BMP-6 | ↑ | ↓ |
| TGF-β₁ | ↑ | [symbol] |
| TGF-β₂ | [symbol] | [symbol] |
| IFN-γ | ↑ | [symbol] |
| TNF-α | ↑ | [symbol] |
| PDGF-A | ↑ | ↓ |
| PDGF-receptor | ↑ | ↓ |
| IL-6 | [symbol] | [symbol] |
| MIF | [symbol] | [symbol] |
| IL-1 | [symbol] | [symbol] |
| IL-12 p35 | [symbol] | [symbol] |

^{a}p < .05 compared to control;
^{b}p < .05 compared to PTH;
Increased, ↑;
no change, [symbol] ;
decreased, ↓

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating a bone condition in a mammal, said method comprising:
a) administering to said mammal an amount of a PDGF signaling antagonist effective to treat said bone condition; and
b) monitoring said bone condition in said mammal.

2. The method of claim 1, wherein said PDGF signaling antagonist is a PDGF receptor antagonist.

3. The method of claim 2, wherein said PDGF receptor antagonist is triazolopyrimidine or a pharmaceutically acceptable salt thereof.

4. The method of claim 1, wherein said bone condition is a metabolic bone condition.

5. The method of claim 4, wherein said metabolic bone condition is primary or secondary osteoporosis.

6. The method of claim 1, wherein said bone condition is parathyroid bone disease.

7. The method of claim 1, wherein monitoring said bone condition comprises monitoring serum calcium levels in a biological sample from said mammal.

8. The method of claim 7, wherein said biological sample is selected from the group consisting of blood, serum, plasma, bone, and urine.

9. The method of claim 1, wherein monitoring said bone condition comprises monitoring levels of a marker of bone turnover in a biological sample from said mammal.

10. The method of claim 9, wherein said marker is selected from the group consisting of osteocalcin, bone specific alkaline phosphatase, type I C-terminal propeptide of type I collagen, deoxypyridinoline, and pyridinoline.

11. The method of claim 1, wherein monitoring said bone condition comprises monitoring bone mass or bone density in said mammal.

12. The method of claim 1, wherein bone mass or bone density is monitored by dual-energy absorptiometry or computed tomography.

13. A method for preventing development of a bone condition in a mammal, said method comprising:
a) administering to said mammal an amount of a PDGF signaling antagonist effective to prevent development of said bone condition; and
b) monitoring said mammal for development of said bone condition.

14. The method of claim 13, wherein said bone condition is osteoporosis.

15. The method of claim 13, wherein said PDGF signaling antagonist is a PDGF receptor antagonist.

16. A method for identifying a triazolopyrimidine derivative for treating a bone condition, said method comprising
a) contacting a cell culture with a derivative of triazolopyrimidine in the presence of PDGF;
b) monitoring production of a matrix protein in said cell culture; and
c) identifying said derivative as suitable for treating said bone condition if production of said matrix protein decreases.

17. The method of claim 16, wherein said matrix protein is osteocalcin.

18. The method of claim 16, wherein said matrix protein is collagen.

19. The method of claim 16, wherein said cell culture is a human bone cell culture.

20. The method of claim 16, wherein said cell culture is a rodent bone cell culture.
